(19) United States
(12) Patent Application Publication
(10) Pub. No.: US 2019/0269365 A1
(43) Pub. Date: Sep. 5, 2019

(54) Dynamic Quantitative Brain Activity Data Collection Devices, Systems, and Methods

(71) Applicant: NewMindVanDrakeChamp LLC, Norcross, GA (US)

(72) Inventor: Richard Soutar, Woodstock, GA (US)

(21) Appl. No.: 16/289,564

(22) Filed: Feb. 28, 2019

Related U.S. Application Data
(60) Provisional application No. 62/637,088, filed on Mar. 1, 2018.

(51) Int. Cl. A61B 5/00; A61B 5/0478

(52) U.S. Cl. CPC A61B 5/6803; A61B 2562/0217; A61B 5/0478

(57) ABSTRACT

In some aspects, the present disclosure relates to devices, systems, and methods for collecting brain activity data. In some embodiments, a head-worn device collects EEG data using a plurality of sensors that are positioned at multiple points across the scalp of the subject. Each of the sensors can be constructed with a housing having an absorbent portion that can be moistened with the conductive fluid. The sensors can be held in position at a subject's head through the use of a framework of flexible bands that are adjustable to accommodate different head sizes.

### CROSS-REFERENCE TO RELATED APPLICATION

This application claims priority to and the benefit of U.S. Provisional patent Application 62/637,088 filed Mar. 1, 2018, which is hereby incorporated by reference herein in its entirety.

### BACKGROUND

The disclosed technology generally relates to collection of brain activity data for use in neurometric analysis.

Some past approaches to brain activity data collection, and in particular electroencephalography (EEG) data collection, involved pasting or taping electrodes to the scalp one by one and measuring the placement carefully using a tape measure and coordinate system as a guide. This process proved very tedious and time consuming, and electrodes can easily fall off or become misplaced. Patients were required to sit still for long periods of time and those who were too young or ill were frequently incapable of enduring the process in a manner that resulted in the collection of quality data. Consequently, EEG signal quality was frequently substandard and resulted in the need to record long records of data to obtain usable segments of sufficient length for analysis.

Flexible caps that could fit over the scalp have been used in some past approaches. Electrodes can be embedded in a cap in a manner that precluded the need for measurement of electrode distances. Such caps need be dried between uses, however, and their electrodes often utilized injected conductive gels that were messy, required a syringe applicator, and were difficult to clean from a patient's hair. These caps were often tight and uncomfortable, sometimes resulting in pain and frequently causing patients to tense up in a manner that resulted in contamination of collected EEG signal with muscle artifact signal. In addition, considerable training was required to learn how to successfully put on the caps in a manner that resulted in an acceptable quality signal. Further, large and expensive arrays of EEG amplifiers were required in some conventional approaches to collect all 19 channels of EEG data at one time.

Other conventional approaches have used a variety of straps for fitting, but these can result in problems with tangled straps and a high number of adjustments being required to fit such a device to a spheroid surface such as the scalp. These designs can have placement accuracy issues and still require different model sizes to accommodate the variety in surface geometry of the scalp across different patients. Still other conventional approaches have used interchangeable electrodes with electrolyte pumping systems in effort to simplify electrolyte application, but such pumping systems can make a cap awkward and bulky and can impose a difficult and time consuming cleaning process. Other approaches have used spring loaded electrodes that puncture the skin for quick electrical connection; these can require complete electrode replacement with each use in order to maintain sterile application and avoid infection. Still other past approaches have used a full helmet rather than a cap. Such helmet devices, however, can be awkward to use in a clinical setting, heavy and difficult to clean, and can confine body heat, generating excessive sweat in many patients that thereby degrades EEG signal quality.

Removable electrodes have been employed by some designers, but with minimal resulting advantages. Electrodes have been very expensive and cap fittings are still uncomfortably tight and laborious to achieve. In addition, multiple cap sizes are still required to accommodate the range of human head sizes.

Some past approaches also have limited amplifier collection options, based on plug connectors associated with each design. Conventional data collection procedures require the use of abrasive gels and solvents like isopropyl alcohol to create the required impedance or resistance measures.

It is with respect to these and other considerations that the various embodiments described below are presented.

### SUMMARY

In some aspects, the present disclosure relates to devices, systems, and methods for collecting data related to neurological activity of the brain of a subject, for example electrical signals corresponding to activity of the brain of the subject, such as electroencephalography (EEG) data. In some embodiments, a head-worn device collects EEG data using a plurality of sensors that are positioned at multiple points across the scalp of the subject. In some embodiments, each of the sensors can be constructed with a housing formed with a reservoir to hold an electrically conductive fluid such as an electrolytic solution, and an absorbent portion disposed within the housing which can be moistened with the conductive fluid. The sensors can be held in position at a subject's head through the use of a framework of flexible bands that are adjustable to accommodate different head sizes. In some embodiments, a headset stand contains a reservoir for conductive fluid which keeps the sensors moist when the headset is mounted on the stand. In some embodiments, an integrated interface or module is configured to make dynamic connections to any combination or permutation of the sensors. A dynamic collection system (DCS) can collect the data from a single sensor or any combination or permutation of sensors such that different areas of the brain can be examined. The sensed data can be transmitted through wired and/or wireless connections for further interface with an EEG amplifier, computer and/or database system for quantifying and analysis purposes.

In one aspect, the present disclosure relates to a device for collecting data corresponding to neurological activity of the brain of a subject. In one embodiment, the device includes a plurality of sensors configured to receive and conduct electrical signals corresponding to neurological activity of the brain of the subject. The device also includes a framework configured to contact and surround predetermined portions of the head of the subject and to secure the plurality of sensors in predetermined positions. Each of the plurality of sensors includes a housing, an absorbent portion positioned at least partially within the housing and configured to absorb an electrically conductive fluid and to contact a surface of the head of the subject, and a conductive member electrically coupled to the absorbent portion. The conductive member is positioned at least partially within the housing and configured to conduct electrical signals from the brain of the subject, through the absorbent portion when the absorbent portion contains the electrically conductive fluid.

In some embodiments, each of the plurality of sensors includes a reservoir portion configured to hold and/or selectively receive the electrically conductive fluid.

In some embodiments, the collected data is electroencephalography (EEG) data.

In some embodiments, the electrically conductive fluid is an electrolytic solution.

In some embodiments, the framework is part of a headset for covering at least a portion of the head of the subject and comprises flexible portions having bands configured to secure the plurality of sensors in the predetermined positions.

In some embodiments, the conductive member is configured to conduct electrical signals from the brain of the subject, through the absorbent portion when the absorbent portion contains the electrically conductive fluid, and to one or more outside data collection systems.

In some embodiments, the conductive member includes at least one metal pin.

In some embodiments, the absorbent portion is biased against the head of the subject, by a biasing member of the sensor, when the framework is positioned on the head of the subject.

In some embodiments, the absorbent portion and/or conductive member are coupled to electrical transmission wires configured to conduct collected electrical signals from the sensors to one or more outside data collection systems.

In some embodiments, the electrical transmission wires are at least partially integrated in one or more portions of the framework.

In some embodiments, the device includes a wireless transmission system, configured to wirelessly transmit collected electrical signals from the sensors to one or more outside data collection systems.

In some embodiments, the sensors are adjustable in a substantially lateral direction along the framework.

In some embodiments, the sensors are adjustable with respect to the framework to move the sensors along an axis running towards and away from the surface of the head of the subject.

In some embodiments, the device includes a mastoid extension portion that extends from at least one band of the framework, for contacting the mastoid region, and which includes at least one reference sensor.

In another aspect, the present disclosure relates to a system for collecting data corresponding to neurological activity of the brain of a subject. In some embodiments, the system includes a head-worn data collection device that includes a plurality of sensors and is configured to receive and conduct electrical signals corresponding to neurological activity of the brain of the subject. The system also includes a dynamic collection system (DCS) that is electrically coupled to the head-worn data collection device and configured to receive the electrical signals from the head-worn data collection device. The DCS is further configured to selectively open or close connections to one or more particular sensors of the plurality of sensors for a predetermined period of time and/or in accordance with a predetermined sequence, to selectively activate or deactivate the respective particular sensors to collect data corresponding to activity at particular regions and/or networks of the brain of the subject.

In some embodiments, the DCS is configured to selectively activate particular sensors of the plurality of sensors simultaneously for collecting data at different locations simultaneously.

In some embodiments, the system also includes a module configured to determine what sensors of the plurality of sensors are actively collecting data.

In some embodiments, the head-worn data collection device includes a framework configured to contact and surround predetermined portions of the head of the subject and to secure the plurality of sensors in predetermined positions. Each of the plurality of sensors includes a housing and an absorbent portion positioned at least partially within the housing and configured to absorb an electrically conductive fluid and to contact a surface of the head of the subject. Each of the sensors also includes a conductive member that is electrically coupled to the absorbent portion and positioned at least partially within the housing. The conductive member is configured to conduct electrical signals from the brain of the subject, through the absorbent portion when the absorbent portion contains the electrically conductive fluid.

In some embodiments, each of the sensors includes a reservoir portion configured to hold and/or selectively receive electrically conductive fluid.

In another aspect, the present disclosure relates to a method for collecting data corresponding to neurological activity of the brain of a subject. In one embodiment, the method includes sensing, by a plurality of sensors of a data collection device, electrical signals corresponding to neurological activity of the brain of the subject, and selectively opening or closing connections to one or more particular sensors of the plurality of sensors for a predetermined period of time and/or in accordance with a predetermined sequence, to selectively activate or deactivate the respective particular sensors to collect data corresponding to activity at particular regions and/or networks of the brain of the subject.

In some embodiments, the method includes selectively activating particular sensors of the plurality of sensors simultaneously for collecting data at different locations simultaneously.

In some embodiments, the method includes determining what sensors of the plurality of sensors are actively collecting data.

In another aspect, the present disclosure relates to a system for collecting data corresponding to neurological activity of the brain of a subject. In one embodiment, the system includes a device for collecting data corresponding to neurological activity of the brain of a subject. The device includes a plurality of sensors configured to receive and conduct electrical signals corresponding to neurological activity of the brain of the subject, and a framework configured to contact and surround predetermined portions of the head of the subject and to secure the plurality of sensors in predetermined positions. Each of the plurality of sensors includes a housing and an absorbent portion positioned at least partially within the housing and configured to absorb an electrically conductive fluid and to contact a surface of the head of the subject. Each of the sensors also includes a conductive member that is electrically coupled to the absorbent portion and positioned at least partially within the housing, and configured to conduct electrical signals from the brain of the subject, through the absorbent portion when the absorbent portion contains the electrically conductive fluid. The system also includes a stand that includes a reservoir configured for holding electrically conductive fluid and engaging with the one or more of the sensors and providing the electrically conductive fluid to the one or more sensors via the reservoir portion of the one or more sensors.

In some embodiments, the stand includes a plurality of receptacles configured to receive respective sensors and configured to provide the electrically conductive fluid to the respective sensors when engaged with the respective sensors.

In some embodiments, the plurality of receptacles are located proximate a top section of the stand and receive the electrically conductive fluid via one or more tubes from a central reservoir of the stand that is proximate the base of the stand.

Other aspects and features according to the present disclosure will become apparent to those of ordinary skill in the art, upon reviewing the following detailed description in conjunction with the accompanying figures.

### BRIEF DESCRIPTION OF THE DRAWINGS

Reference will now be made to the accompanying drawings, which are not necessarily drawn to scale.

FIG. 1 illustrates a brain activity data collection device in accordance with one embodiment.

FIG. 2 illustrates a sensor of the device shown in FIG. 1.

FIG. 3 illustrates another embodiment of a brain activity data collection device, having a mastoid sensor extension.

FIG. 4 illustrates another embodiment of a brain activity data collection device.

FIG. 5A illustrates a conductive pin and an absorbent portion for a sensor in accordance with one embodiment.

FIG. 5B illustrates the conductive pin of FIG. 5A engaging absorbent material, shown outside of a sensor housing.

FIG. 6 illustrates components of a sensor and connective wires with a washer for connecting to a conductive pin.

FIG. 7 illustrates an exploded view of components of a sensor in accordance with one embodiment.

FIG. 8A illustrates a cross-sectional side view of a band and sensor according another embodiment, in a first state.

FIG. 8B illustrates a cross-sectional side view of the band and sensor of FIG. 8A, in a second state.

FIG. 8C illustrates an exploded, cross-sectional side view of the components of the band and sensor shown in FIGS. 8A and 8B.

FIG. 8D illustrates an exploded, isometric view of the components of the band and sensor shown in FIGS. 8A and 8B.

FIG. 9 illustrates a dynamic collection system (DCS) according to one embodiment.

FIG. 10 illustrates a headset stand according to one embodiment with a reservoir system for providing electrically conductive liquid to sensors of a headset for collection of brain activity data.

### DETAILED DESCRIPTION

The disclosed technology, in some aspects, relates to collection of data relating to activity of the brain of a subject, for example electrical signals corresponding to neurological activity of the brain of the subject, such as EEG data.

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

As discussed herein, a “subject” or “patient” generally refers to any applicable human, animal, or other living organism with active brain function for which data relating to neurological activity may be collected, for example electroencephalography (EEG) data.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

A detailed description of aspects of the present disclosure will now be provided with reference to the accompanying drawings. The drawings form a part hereof and show, by way of illustration, specific embodiments or examples. In referring to the drawings, like numerals represent like elements throughout the several figures.

The disclosed technology relates in some aspects to head-worn devices (also referred to herein in some instances as a “cap” or “headset”) that collect data corresponding to electrical activity of the brain of the subject, for example EEG data, using a plurality of sensors that are positioned at multiple points across the scalp of the subject. In some embodiments, each of the sensors can be constructed with a housing formed with a reservoir to hold an electrically conductive fluid such as an electrolytic solution, and an absorbent portion disposed within the housing which can be moistened with the conductive fluid. The sensors can be held in position at a subject's head through the use of a framework of flexible bands and/or as part of a flexible cap, adjustable to accommodate different head sizes and to receivably secure and hold the sensors in different arrangements, such as particular arrays to align with specific parts of the brain as desired. In some embodiments, a headset stand contains a reservoir for conductive fluid which keeps the sensors moist (e.g., through a natural wicking process) when the headset is mounted on the stand.

In some embodiments, an integrated interface or module is configured to make dynamic connections to any combination or permutation of the sensors. By collecting the data from a single sensor or any combination or permutation of sensors, different areas of the brain can be examined. The sensed data can be transmitted through wired and/or wireless connections for further interface with an EEG amplifier, computer and/or database system for quantifying and analysis purposes.

Among other benefits and advantages provided by implementing various aspects of the disclosed technology, through the use of the absorbent material with the electrically conductive fluid, the sensors can feel almost dry to the touch yet have comparable conductivity to wet sensors. The hair is not subjected to oily residue as in the case of conventional approaches that require pastes or gels. Use of the electrically conductive fluid reduces both preparation time and post data collection cleaning procedures in comparison to conventional approaches, and can enhance the conductivity of the sensors with minimal user involvement. Head-worn devices according to embodiments of the disclosed technology described herein are simple to use and can be placed on the subject's head and used to train in a matter of seconds. High quality connections are enabled, with low impedance and a strong signal. Further, some embodiments are passive and require no active amplification for signal collection.

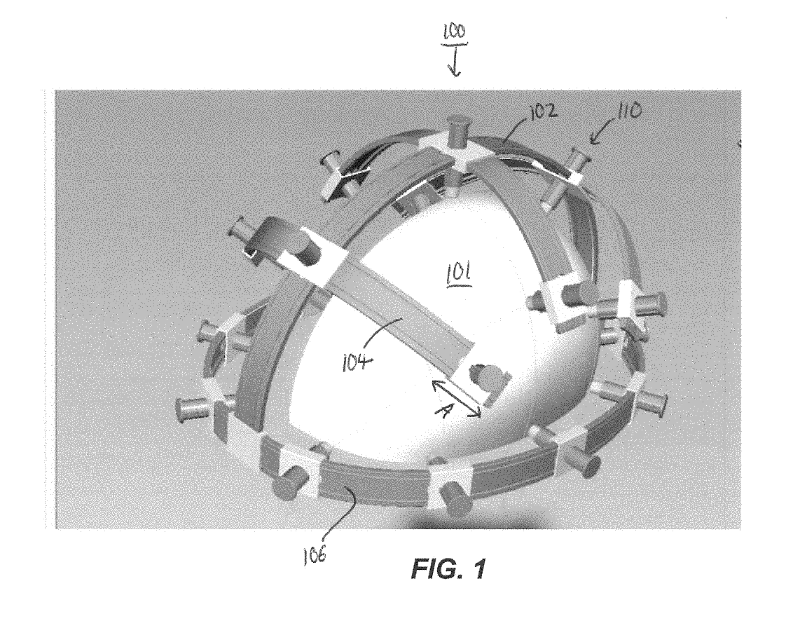

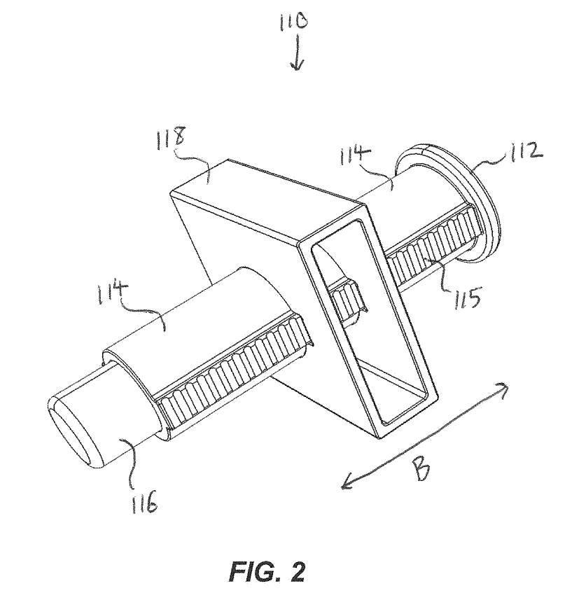

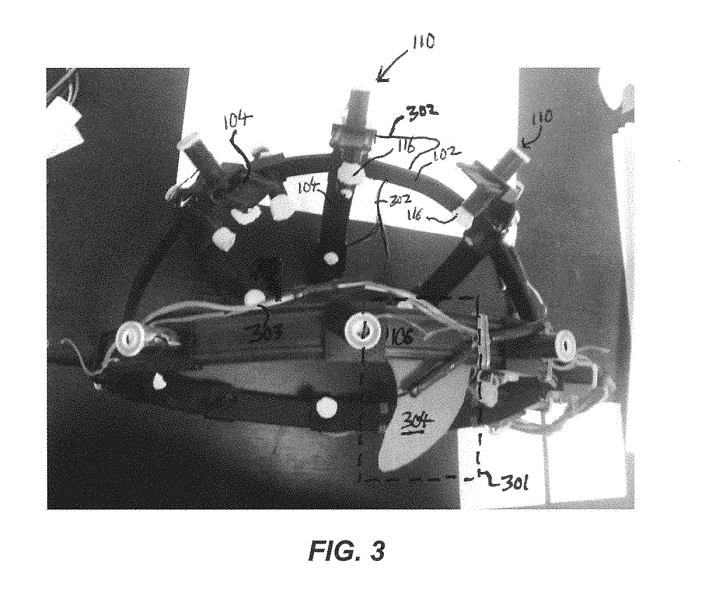

As shown in FIGS. 1 and 2, a head-worn brain activity data collection device 100 includes a framework comprised of bands 102, 104, 106 with a plurality of sensors 110. The bands 102, 104, 106 are adjustable to fit around the head of a subject (see semi-spherical contoured rendering 101, representing a surface, e.g., scalp, of a portion of the head of a subject). The device 100 as shown has bands 106 that run circumferentially around the head as well as bands 102 that run over the top of the head from one point on the circumferential band 106 to the other side, and bands 104 that can run along orientations that are substantially perpendicular to the bands 102. The device 300 of FIG. 3 includes similar bands having these respective arrangements and function (see similarly labeled bands 102, 104, 106). In some embodiments, the sensors 110 can lock into a wide range of positions within the framework of the device 100. The sensors 110 are moveable along the bands of the framework to adjust for the correct positioning. A sensor 110 can adjust up and down (for example along axis B shown in FIG. 2) from the framework of the device 100, with respect to depth (i.e., closer towards or farther away from the surface of the subject's head), which may be facilitated by ribbed or ridged portions 115 of the housing 114. The adjustability facilitates increase or decrease of the overall surface collection area and distance from the scalp for various head sizes. The device 300 shown in FIG. 3 also includes similarly constructed sensors 110.

In some embodiments (for example those shown in FIGS. 1-3), a sensor 110 is also moveable in a substantially lateral direction (for example, along axis A shown in FIG. 1) along bands 102, 104, 106 of the framework of the device, to adjust the distance from the midline of the human head. The lateral-type movement can be facilitated by component 118, shown as a substantially open, rectangular member configured to receive and slide along a band. Since smaller head sizes have a shorter distance from the midline of the head, sensor 110 has the mobility to come in closer to the midline as well as move away from the midline for larger head sizes. The adjustability facilitates desired placement for collecting brain activity data (e.g., EEG data) at particular locations, for example at one or more of the 10-20 international standard electrode locations.

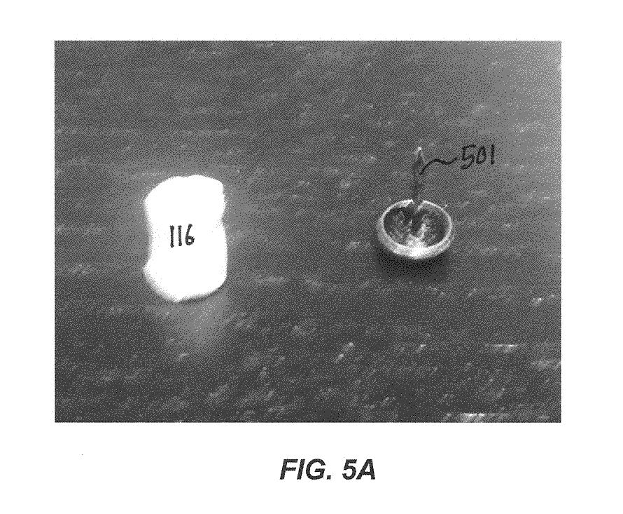

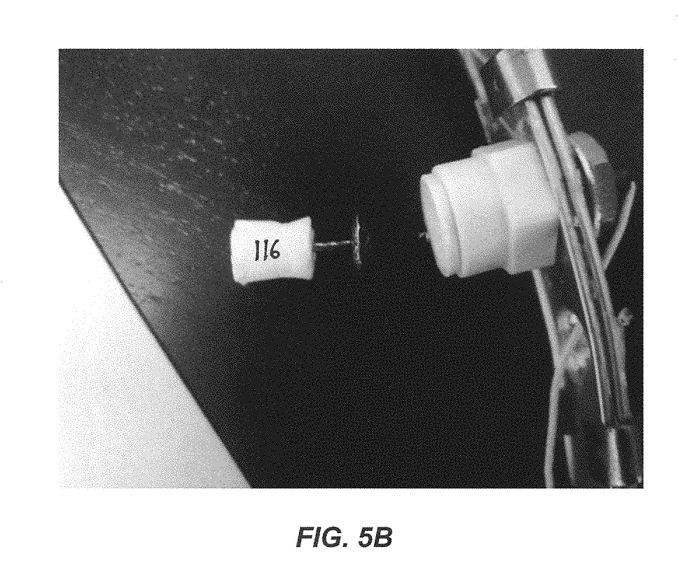

In some embodiments, a sensor is comprised of a housing containing an absorbent material, a conductor which can be in the form of an insertable and replaceable conductive member such as a metal pin (for example, pin 501 shown in FIGS. 5A and 5B), and a supportive solution reservoir. For example, the sensors 110 shown in FIGS. 1-3 are each comprised of a cylindrical housing 114 and a top portion 112 at one end of the housing that can be configured to receive and/or hold (such as by a reservoir portion) an electrically conductive fluid. The conductive fluid (e.g., electrolytic solution, which may be or include a saline solution) may be provided to the sensor 110 by opening of the top portion 112, which may be partially or fully removable or have a rubber covering configured to allow injection of the fluid. The housing 114 can be made of a lightweight material such as plastic and may have a cylindrical shape. In the embodiments of FIGS. 1-3, the housing 114 encompasses an absorbent portion 116 formed from the absorbent material (partially shown in FIG. 2 and FIG. 3) for absorbing the conductive fluid such that the absorbent portion 116 thereby contains the conductive fluid. At the opposite end of the sensor 110 from the top portion 112, part of the absorbent portion 116 (labeled in FIGS. 2 and 3) extends out from the housing 114 and, in use, can contact the scalp of the subject at a desired location. Sensors with a generally cylindrical shape in embodiments described herein may be referred to as reservoir cylinder electrodes (RCEs). In one example, the cylinder can have dimensions of ½ in. diameter and ¾ in. length.

The data collection device 300 shown in FIG. 3 shares common components as those shown for the device 100 of FIGS. 1 and 2 (like components in FIG. 3 share like reference numbers with FIGS. 1-2), but the embodiment of FIG. 3 includes a mastoid extension portion 301. The extension portion 301 includes a section 304 that extends down below the main horizontal band 106 to wrap around the back of the ear (not shown) of the subject to make contact with the mastoid region. The section 304 has one or more sensors (not shown) that serve as reference sensors which can establish reference signal values for comparison with collected brain activity data from sensors of other, main portions of the device 300. Although one extension portion 301 is visible in the view of FIG. 3, the device 300 also includes a corresponding, similar extension portion with one or more reference sensors on the other, opposite side of the band 106 corresponding to the other ear of the subject.

Now referring to embodiments of a sensor in further detail, the sensor housing contains the absorbent portion for enhanced data collection. The absorbent portion can be comprised of a foam-type material that can be moistened by the electrically conductive fluid in order to maintain a high conductivity when in contact with the skin while also efficiently retaining the fluid. The absorbent portion can be manufactured from biocomponent synthetic fiber that can be engineered to meet various density, permeability, and wicking performance requirements, as well as a wide range of molding processes; for example, a polyethylene sheath with a polyester core PET (polyester terephthalate) or a polyester sheath with a PET core, depending on strength, temperature, and molding requirements. The absorbent material may be 11 mm fiber carwick from Porex Corporation, for example. In some embodiments the housing has a small waterproof covering to serve as a reservoir for the conductive fluid. The covering can have a rubber injection point through which a needle and syringe can be used to inject the conductive liquid periodically or as needed. The housing may additionally or alternatively have an opening for introducing the conductive fluid. Alternatively, the electrically conductive fluid may be applied directly to the absorbent portion to be absorbed, for example by placing the absorbent portion into an external container holding such fluid.

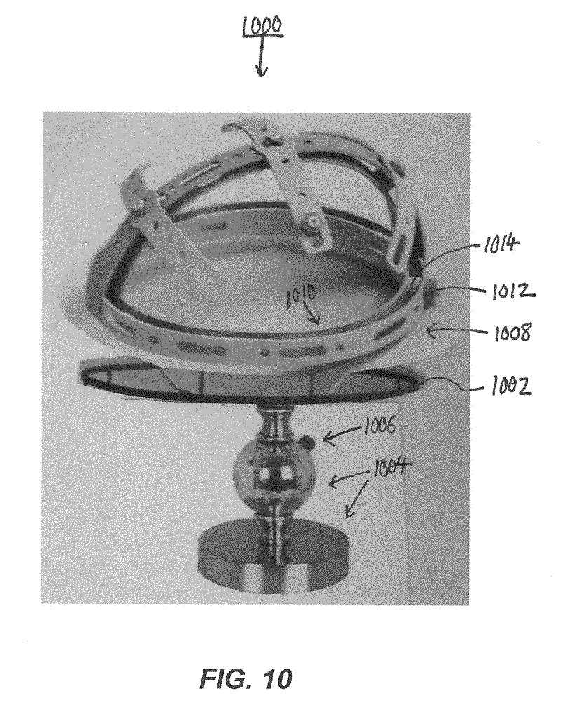

In use, the absorbent material of the absorbent portion is moistened with the electrically conductive fluid to provide constant contact and electrical communication between the point of scalp contact and conductive member(s) inside the housing. In some embodiments, the absorbent material forming the absorbent portion can be placed into the housing by pushing the material into the housing to be held in place by friction. The conductive members can be inserts such as tin pins (see, for example, pin 501 in FIGS. 5A-5B), and may be separable from or integral with the sensor housing. As shown in FIG. 10 and described in further detail below, in some embodiments a headset stand contains a reservoir for conductive fluid, which keeps the sensors moist (e.g., through a natural wicking process) when the headset is mounted on the stand.

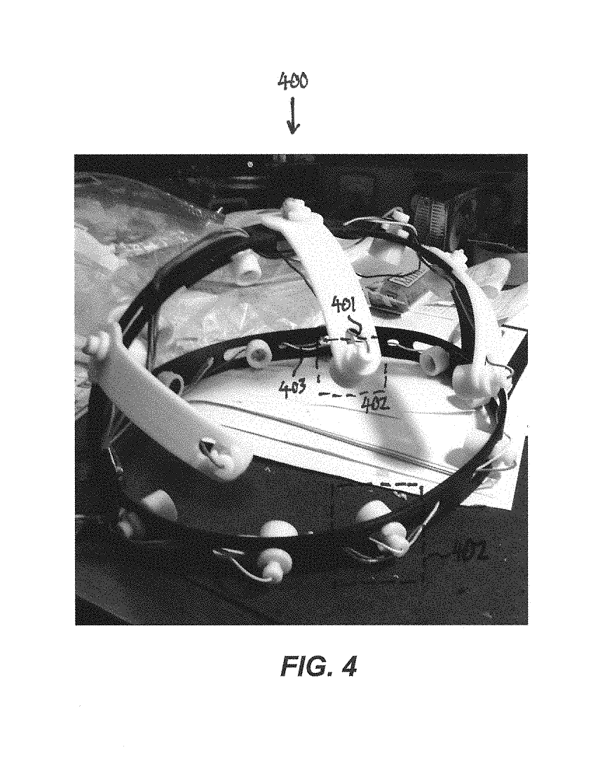

In some embodiments, the conductive members can be made of tin or another suitable conductor that are insertable such that they are easily replaceable and can be repositioned as needed. The conductive member is disposed with respect to the housing such that it contacts the absorbent portion which, when moistened by the electrically conductive fluid, provides for electrical activity to be sensed at a surface of the scalp and the corresponding electrical signal to travel through the absorbent material, through the conductive member, and out of the sensor via sensor wires (e.g., conductive leads or other transmission wires, as shown for example as wires 302 in FIG. 3 and wires 401 in FIG. 4) that are electrically coupled to the conductive member. For example, the sensor wire(s) may be fixed to pin conductors. Additional wiring (e.g., wires 303 in FIG. 3 and wires 403 in FIG. 4) can connect sensors to one another throughout the framework, for example to allow communication of signals for selective control and coordination of the various sensors. Such wiring can run within or be embedded in the headset (e.g., along or in a band of the headset), for example as shown in FIG. 4. The sensor wire(s) can be fixed to the conductive member and sensor housing by, for example, a screw or other fastener that connects into the housing.

The sensor wires can be coupled to a transmission apparatus, such as wired or wireless transmitting systems, to send the signals to be amplified and then digitized by software. In some embodiments, the collected signals are thus transmitted to a collection system which in some embodiments is referred to as a dynamic collection system (DCS), as will be described in further detail below.

FIG. 4 illustrates another embodiment of a brain activity data collection device 400. As shown, the device 400 has several bands similar in arrangement and construction to the bands shown in the embodiments of FIGS. 1 and 3, for adjusting and securing the device on the head of the subject. A plurality of sensors are positioned on the bands. Partial constructions 402 of the sensors are shown, which generally include the housing and the sensor wires 401. Conductive members or absorbent portions are not shown. FIG. 5B shows a configuration with a conductive member 501 (pin) inserted in an absorbent portion 116, outside of and not inserted within the housing of a sensor.

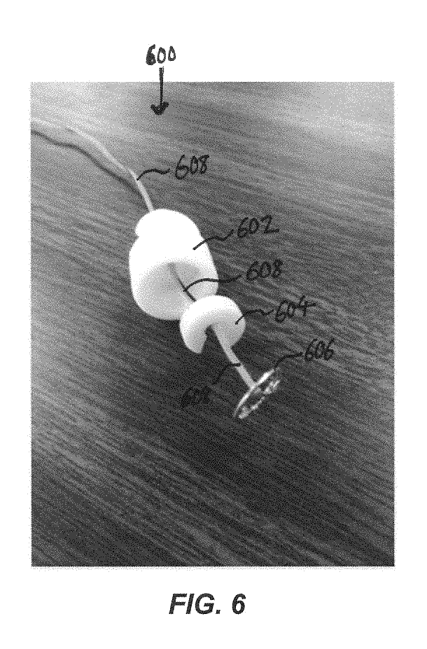

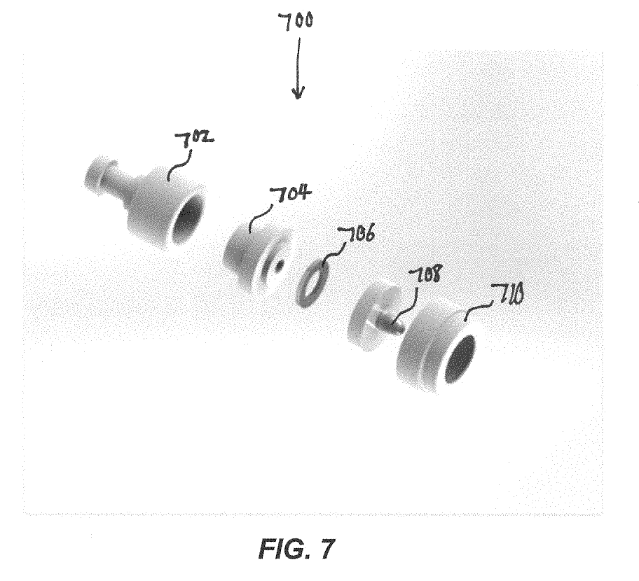

FIG. 6 illustrates components (collectively 600) of a sensor and a connective wire with a washer for connecting to a conductive pin. As shown, a connective wire 608 extends through a housing portion 602 and a reservoir portion 604 and connects at one end to a conductive washer 606. In use, the washer 606 can be connected to a conductive member such as a metal pin (see, e.g., pin 501 in FIGS. 5A and 5B) of a sensor. FIG. 7 illustrates an exploded view of a sensor 700 according to one embodiment. As shown, the components of the sensor 700 include one end portion 702 of the sensor housing, a sensor reservoir 704, a washer 706 that a conductive wire attaches to, a conductive member 708 that is inserted into an absorbent material (not shown), and a section 710 that holds the absorbent material.

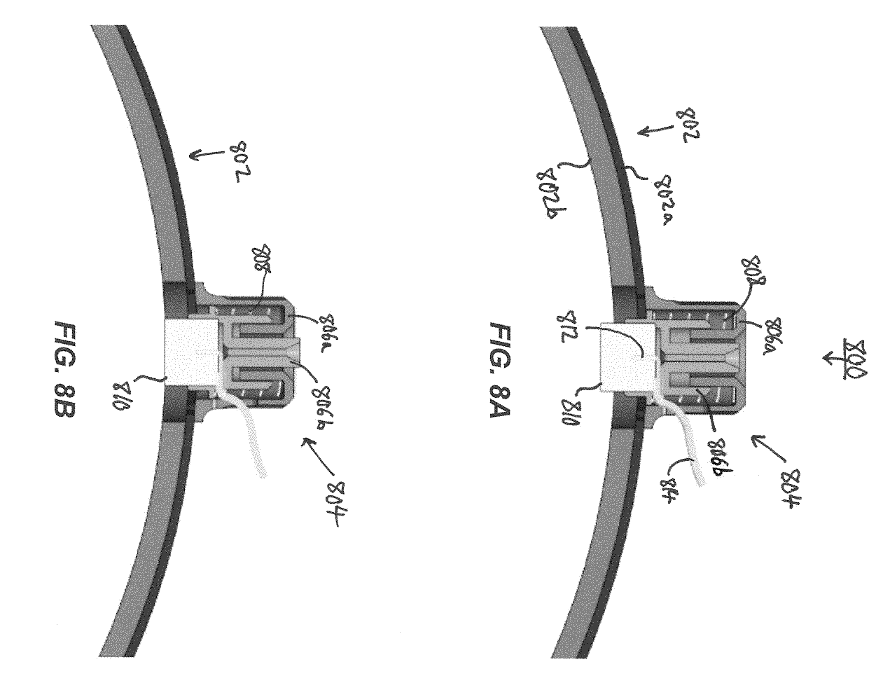

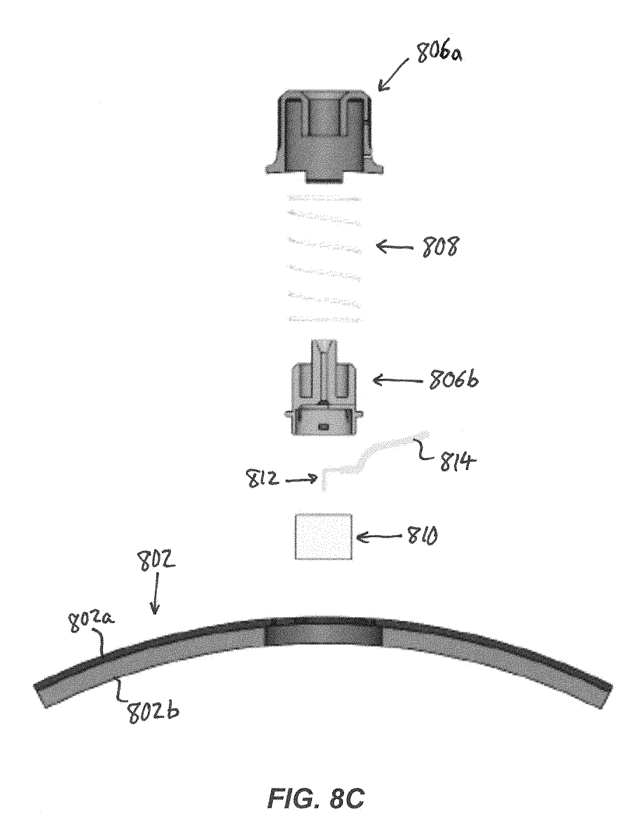

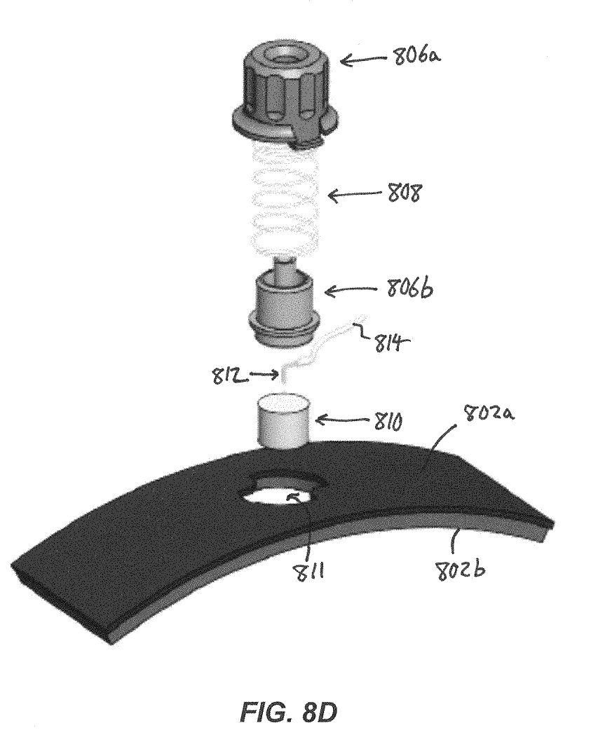

FIGS. 8A and 8B show cross-sectional, side views of a flexible band 802 and sensor 804 (collectively 800) according to another embodiment, which can be used in a framework comprised of bands and sensors in a head-worn brain activity data collection device. Similar to the bands shown in the embodiments of FIG. 1 and FIG. 3, the band 802 shown in FIGS. 8A-8D is adjustable to fit around at least a portion of the head of a subject (see, e.g., rendering of head 101 in FIG. 1). The band 802 shown in FIGS. 8A-8D is a representative band, with a representative sensor 804, which can be part of a plurality of bands and sensors that may run circumferentially around the head of the subject and/or over the top of the head of the subject, among other alignments, to position the bands and sensors on the head of the subject as desired. Adjustability of the bands facilitates desired placement for collecting brain activity (e.g., EEG data) at particular locations and accommodates variance in head dimensions across different subjects.

FIG. 8A illustrates the band 802 and sensor 804 in an initial state wherein the band 802 and sensor 804 are not being worn by a subject. FIG. 8B illustrates the band 802 and sensor 804 in a state wherein the band 802 would be placed against the subject's head, causing a displacement of the inner housing portion 804b and connected absorbent portion 810 of the sensor 804 in a direction away from the head of the subject, due to force applied with respect to a bias member 808, while also keeping the band 802 and absorbent portion 810 of the sensor 804 in contact with the scalp of the head of the subject at a desired location. FIG. 8C illustrates an exploded view of the components of the band 802 and sensor 804 shown in FIGS. 8A and 8B, from a cross-sectional side view, and FIG. 8D illustrates an exploded, isometric view of the components of the band 802 and sensor 804 shown in FIGS. 8A and 8B.

The band 802 is flexible and is configured to conform to the shape of the head of the subject. An inner portion 802b of the band that, in use, directly contacts the subject's head, is comprised of foam lining or other suitable material configured to provide for a comfortable distribution of force. As shown, the sensor 804 has a housing 806 formed with an inner housing portion 806b and outer housing portion 806a surrounding the inner housing portion 806b. The outer housing portion 806a mounts to the band 802. A biasing member 808, here shown as a compression spring, is engaged to the inner housing portion 806b and biases the inner housing portion 806b such that, in the initial state shown in FIG. 8A, the inner housing portion 806b rests at one end against an outer portion 802a of the band 802 and the absorbent portion 810 at least partially extends outwards towards the subject's scalp. The absorbent portion 810 at least partially extends through an opening 811 defined through the band 802 (see particularly FIG. 8C). The absorbent portion 810 extends through the defined opening 811 towards, and presses against, the subject's scalp when in use.

As shown, from the initial position of the inner housing portion 806b in FIG. 8A to the position of the inner housing portion 806b in FIG. 8B, in FIG. 8B the inner housing portion 806b has moved in a direction away from the subject's head and relative to the outer housing portion 806a which remains fixed, due to the relative force between the band 802 and sensor 804 being directly in contact with and against the head of the subject, thereby generating a force that acts against the normal bias of the bias member 808 (e.g., about 1 N) and displacing the inner housing portion 806b as shown. In use, the absorbent material of the absorbent portion 810 is moistened with an electrically conductive fluid to provide constant contact and electrical communication between the point of scalp contact and conductive member(s). The sensor 804 includes one or more conductive members (see, e.g., conductive member 812) protruding into the absorbent portion, and a sensor wire 814 is electrically connected to the conductive member 812.

Use of a dynamic collection system (DCS) in accordance with some embodiments improves the function of EEG amplifiers, such that any amplifier with at least one available channel for EEG collection can be used to collect EEG data. This is accomplished by a timing mechanism that facilitates the collection of any combination or permutation of EEG data from any sensor on the scalp for any combination or permutation of time. For example, data from one sensor may be collected for 500 milliseconds and next data from the next sensor is collected for the same amount of time or any permutation of time. The sequence can remain static or dynamic depending on the EEG collection software. The DCS can also be used with any EEG amplifier with more than one available channel. This EEG data collection method of using any combination or permutation from the sensors on the subject's head, in order to specifically target different regions and networks of the brain, can significantly enhance quantitative analysis.

Since different brain regions send information to and from other areas of the brain, it is important to be able to collect data from these regions with precision. Sampling data from predetermined brain regions provides information about directional flow and timing with respect to connectivity. Using a DCS in accordance with certain embodiments allows for a one or two channel EEG recording device to collect data from many sites by opening the connections to any sensor for a predetermined amount of time. This can be accomplished by connecting any sensors to a module that determines which sensors are actively collecting data.

Accordingly, specific groups of sensors can allow the acquisition of EEG signals in sequential groups that can be later aggregated and integrated during an analysis phase. This represents an advance that reduces the need for large aggregates of EEG amplifiers and allows for the substitution of two to four amplifiers at a significantly lower cost affordable to smaller independent users. Among other advantages, this enhances the reach of the technology, simplifies its employment and consequently benefits a larger audience.

In some embodiments, an interface is provided for a wide variety of single or dual channel EEG amplifiers with the use of a switchbox that allows sequential collection of standard 19 channel 10-20 sites. The switchbox can be part of an integrated interface with the headset as described in accordance with various embodiments above, with various available amplifiers. The switchbox can employ a solenoid driven switch that connects an EEG amplifier with each sensor location in sequence. Samples of various combinations of locations can be collected at different time intervals as designated by an external computer driven program or in a standard fixed sequence of locations by static design and triggered by a single button switch.

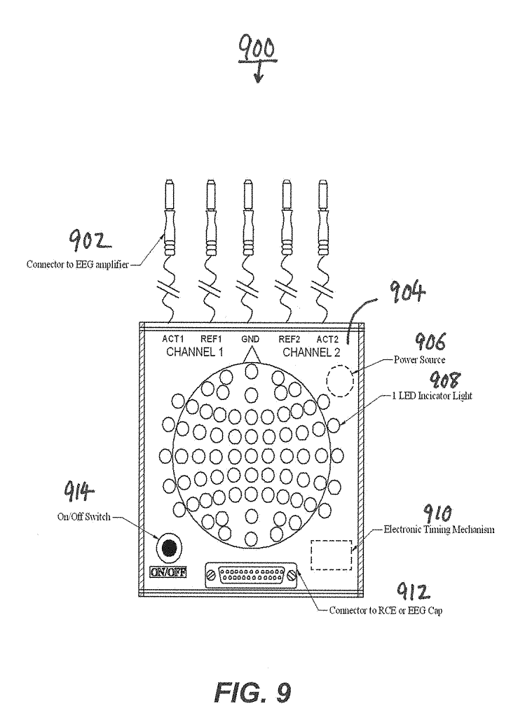

FIG. 9 shows a DCS 900 in accordance with one embodiment. The DCS 900 includes an electronic timing mechanism 910 that can selectively open and close connections in any combination or permutation to available EEG sensors on the subject's head. The DCS 900 also includes connectors 902 to connect the module 904 to an EEG amplifier, and connectors (see connector 912) to connect the module 904 to RCEs or an EEG cap. Also shown are a power source 906 in the module 904, an on/off switch 914, and LED indicator lights 908 which can provide an indication of one or more sensors that are active for collecting EEG data.

FIG. 10 illustrates a headset stand 1000 in accordance with one embodiment. The headset stand contains a reservoir for electrically conductive fluid such as an electrolytic solution, which keeps the sensors moist when the headset is mounted on the stand. In the embodiment shown in FIG. 10, the headset stand 1000 includes a reservoir section 1004 for holding conductive fluid, which is accessible from the outside by way of a screw-on cap 1006 that provides a point for adding conductive fluid. Supported above the reservoir section 1004 is a ring-like section 1002 that includes fine plastic channels. As shown, a main cap 1008 for collecting brain activity data is mated with a corresponding inner cap 1010 such that male portions of the sensors 1012 of the main cap 1008 mate with female receptacles (see, e.g., partial view of element 1014) of the inner cap 1010. The main cap 1008 can be constructed with a framework of bands, sensors, wires, etc. that uses some or all of the components and has the same or similar construction as the components of the headset/devices shown in the embodiments of one or more of FIGS. 1-4. For example, the bands of the main cap 1008 may be the same as or similar to the bands 102, 104, 106 of the device 100 shown in FIG. 1, and the sensors 1012 of the main cap 1008 may be the same as or similar to the sensors 110 shown in FIG. 1). Flexible tubes (not shown) extend through supporting sections of the stand 1000 and are in fluid communication with the reservoir section 1004. When the main cap 1008 is mated with the inner cap 1010 and rested on the ring-like section 1002, tubes that connect to the female receptacles of the inner cap 1010 thereby provide the conductive fluid to the sensors 1012 of the main cap 1008.

In one embodiment, the headset stand has a central reservoir with plastic tubes that connect to a plurality of cylinders (e.g., 23 cylinders, each ½ in. diameter) that house each sensor individually when the cap is placed on the headset stand. The stand can be made of a plastic or other suitable material and can have a round base and a cylindrical support post. For example, in one embodiment, the stand has a round base 6 in. diameter and ¾ in. thickness, with a cylindrical support post ¾ in. in diameter extending upward for 8 inches. A half plastic sphere member that is substantially oval in shape and which fits inside the cap, is mounted on top of the support post. The half sphere is covered with cylindrical receptacles distributed in a manner to receive all of the cap sensors when the cap is mounted on the stand. Flexible plastic tubes extend down from each sensor through the hollow support post and into the hollow base of the stand that contains the reservoir.

Utilizing the above-described embodiments of a headset stand, once the sensors are recharged by absorbing the conductive fluid using the headset stand, the absorbent material of the sensors can remain conductive for 8-12 hours or more, depending on factors such as the surrounding humidity.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. The patentable scope of certain embodiments of the disclosed technology is indicated by the appended claims, rather than the foregoing description, and all changes that come within the meaning and range of equivalents thereof are intended to be embraced therein.

What is claimed is:

1. A device for collecting data corresponding to neurological activity of the brain of a subject, comprising:
a plurality of sensors configured to receive and conduct electrical signals corresponding to neurological activity of the brain of the subject;
a framework configured to contact and surround predetermined portions of the head of the subject and to secure the plurality of sensors in predetermined positions,
wherein each of the plurality of sensors comprises:
a housing,
an absorbent portion positioned at least partially within the housing and configured to absorb an electrically conductive fluid and to contact a surface of the head of the subject, and
a conductive member electrically coupled to the absorbent portion and positioned at least partially within the housing, and configured to conduct electrical signals from the brain of the subject, through the absorbent portion when the absorbent portion contains the electrically conductive fluid.

2. The device of claim 1, wherein each of the plurality of sensors further comprises a reservoir portion configured to hold and/or selectively receive the electrically conductive fluid.

3. The device of claim 1, wherein the collected data is electroencephalography (EEG) data.

4. The device of claim 1, wherein the electrically conductive fluid is an electrolytic solution.

5. The device of claim 1, wherein the framework is part of a headset for covering at least a portion of the head of the subject and comprises flexible portions having bands configured to secure the plurality of sensors in the predetermined positions.

6. The device of claim 1, wherein the conductive member is configured to conduct electrical signals from the brain of the subject, through the absorbent portion when the absorbent portion contains the electrically conductive fluid, and to one or more outside data collection systems.

7. The device of claim 1, wherein the conductive member comprises at least one metal pin.

8. The device of claim 1, wherein the absorbent portion is biased against the head of the subject by a biasing member of the sensor when the framework is positioned on the head of the subject.

9. The device of claim 1, wherein the absorbent portion and/or conductive member are coupled to electrical transmission wires configured to conduct collected electrical signals from the sensors to one or more outside data collection systems.

10. The device of claim 9, wherein the electrical transmission wires are at least partially integrated in one or more portions of the framework.

11. The device of claim 1, further comprising a wireless transmission system, configured to wirelessly transmit collected electrical signals from the sensors to one or more outside data collection systems.

12. The device of claim 1, wherein the sensors are adjustable in a substantially lateral direction along the framework.

13. The device of claim 1, wherein the sensors are adjustable with respect to the framework to move the sensors along an axis running towards and away from the surface of the head of the subject.

14. The device of claim 1, further comprising a mastoid extension portion extending from at least one band of the framework for contacting the mastoid region, and comprising at least one reference sensor.

15. A system for collecting data corresponding to neurological activity of the brain of a subject, comprising:
a head-worn data collection device comprising a plurality of sensors and configured to receive and conduct electrical signals corresponding to neurological activity of the brain of the subject; and
a dynamic collection system (DCS) electrically coupled to the head-worn data collection device and configured to receive the electrical signals from the head-worn data collection device, and wherein the DCS is further configured to selectively open or close connections to one or more particular sensors of the plurality of sensors for a predetermined period of time and/or in accordance with a predetermined sequence, to selectively activate or deactivate the respective particular sensors to collect data corresponding to activity at particular regions and/or networks of the brain of the subject.

16. The system of claim 15, wherein the DCS is configured to selectively activate particular sensors of the plurality of sensors simultaneously for collecting data at different locations simultaneously.

17. The system of claim 16, further comprising a module configured to determine what sensors of the plurality of sensors are actively collecting data.

18. The system of claim 15, wherein the head-worn data collection device comprises a framework configured to contact and surround predetermined portions of the head of the subject and to secure the plurality of sensors in predetermined positions, and wherein each of the plurality of sensors comprises:
a housing,
an absorbent portion positioned at least partially within the housing and configured to absorb an electrically conductive fluid and to contact a surface of the head of the subject, and
a conductive member electrically coupled to the absorbent portion and positioned at least partially within the housing, and configured to conduct electrical signals from the brain of the subject through the absorbent portion when the absorbent portion contains the electrically conductive fluid.

19. The system of claim 18, wherein each of the plurality of sensors further comprises a reservoir portion configured to hold and/or selectively receive electrically conductive fluid.

20. A method for collecting data corresponding to neurological activity of the brain of a subject, comprising:
sensing, by a plurality of sensors of a data collection device, electrical signals corresponding to neurological activity of the brain of the subject;
selectively opening or closing connections to one or more particular sensors of the plurality of sensors for a predetermined period of time and/or in accordance with a predetermined sequence, to selectively activate or deactivate the respective particular sensors to collect data corresponding to activity at particular regions and/or networks of the brain of the subject.

21. The method of claim 20, comprising selectively activating particular sensors of the plurality of sensors simultaneously for collecting data at different locations simultaneously.

22. The method of claim 20, further comprising determining what sensors of the plurality of sensors are actively collecting data.

23. A system for collecting data corresponding to neurological activity of the brain of a subject, comprising:
a device for collecting data corresponding to neurological activity of the brain of a subject, comprising:
a plurality of sensors configured to receive and conduct electrical signals corresponding to neurological activity of the brain of the subject;
a framework configured to contact and surround predetermined portions of the head of the subject and to secure the plurality of sensors in predetermined positions,
wherein each of the plurality of sensors comprises
a housing,
an absorbent portion positioned at least partially within the housing and configured to absorb an electrically conductive fluid and to contact a surface of the head of the subject, and
a conductive member electrically coupled to the absorbent portion and positioned at least partially within the housing, and configured to conduct electrical signals from the brain of the subject, through the absorbent portion when the absorbent portion contains the electrically conductive fluid; and
a stand comprising a reservoir configured for holding electrically conductive fluid and engaging with the one or more of the sensors and providing the electrically conductive fluid to the one or more sensors.

24. The system of claim 23, wherein the stand comprises a plurality of receptacles configured to receive respective sensors and configured to provide the electrically conductive fluid to the respective sensors when engaged with the respective sensors.

25. The system of claim 24, wherein the plurality of receptacles are located proximate a top section of the stand and receive the electrically conductive fluid via one or more tubes from a central reservoir of the stand that is proximate the base of the stand.
